(19) United States
(12) Patent Application Publication
(10) Pub. No.: US 2016/0192974 A1
(43) Pub. Date: Jul. 7, 2016

(54) ULTRASONIC DEPTH GAUGE

(71) Applicant: Michael Clain, Riverside, CT (US)

(72) Inventor: Michael Clain, Riverside, CT (US)

(21) Appl. No.: 14/975,278

(22) Filed: Dec. 18, 2015

Related U.S. Application Data
(60) Provisional application No. 62/099,417, filed on Jan. 2, 2015.

(51) Int. Cl. A61B 17/88; A61B 8/08; A61B 17/56; A61B 8/12

(52) U.S. Cl. CPC A61B 17/8872; A61B 8/12; A61B 8/0875; A61B 17/56; A61B 2017/90

(57) ABSTRACT

Systems and methods for providing an ultrasonic depth gauge. The depth gauge accurately measures the depth of a drill hole in a bone using ultrasonic waves so that an appropriately-sized bone screw may be selected. The ultrasonic depth gauge includes a probe that is selectively insertable and extendable into a drill hole of a bone. The probe includes an ultrasonic transducer positioned at a distal end that is operative to detect when the probe has extended through the entire drill hole by detecting changes in echo signals. The gauge also includes a sensor for measuring the length of the probe inside of the drill hole when the probe is extending through the entire depth of the drill hole. Using this measured length, the depth of the drill hole may be accurately determined.

### BACKGROUND OF THE INVENTION

1. Field of the Invention

The invention described herein is directed generally to systems and methods for measuring length or depth, and more specifically, to systems and methods for measuring the depth of a drill hole in a bone during surgery using ultrasonic waves.

2. Description of the Related Art

Bone screws are often used by orthopedic surgeons during the repair of injuries, for example, bone fractures. Bone screws may be screwed into a drill hole made in a bone that extends from a first bone wall (cortex), through the bone, and through a second bone wall (cortex) opposite the first bone wall. As can be appreciated, the tip of a bone screw should not protrude excessively past the end of the drill hole and into the surrounding soft-tissue because this may result in irritation or damage to the soft-tissue. As a result, it is important for a surgeon to know the precise depth of the drill hole so that a bone screw having an appropriate length may be selected and used.

In previous devices for measuring the depth of a drill hole in a bone, a piston with a hook is introduced though a drill hole in a bone and the hook engages an opposite bone wall (cortex) on the far side of the drill hole. Then, a measuring sleeve is shifted on the piston in the direction of the bone until the measuring sleeve is in contact with the bone surface on the near side of the drill hole, such that the distance between the measuring sleeve and the hook represents the depth of the drill hole. However, this method of measuring the depth of the drill hole often results in inaccurate measurements. As an example, the hook may slip off the opposite bone wall (cortex) and displacement of the measuring sleeve may pull the hook into the bone, resulting in an incorrect measurement and, ultimately, selection of bone screw having an incorrect length. Selecting a bone screw having an incorrect length can have various undesirable consequences, including additional pain or discomfort for the patient, irritation or damage to soft-tissue, additional cost for discarded bone screws, and may lead to more complicated or additional surgeries.

### BRIEF DESCRIPTION OF THE SEVERAL VIEWS OF THE DRAWINGS

FIG. 1 is a front elevational view of an ultrasonic depth gauge in accordance with an embodiment of the invention when in a retracted position.

FIG. 2 is a front elevational view of the ultrasonic depth gauge of FIG. 1 when in an extended position.

FIG. 3 is a partial cross-sectional view of the ultrasonic depth gauge of FIG. 1.

FIG. 4 is a block diagram of the ultrasonic depth gauge of FIG. 1.

### DETAILED DESCRIPTION OF THE INVENTION

Embodiments of the invention described herein are directed to systems and methods for accurately measuring the depth of a drill hole in a bone using ultrasonic waves. Generally, embodiments of the invention operate by providing a probe that is selectively insertable into a drill hole of a bone and, using ultrasonic waves, measuring the length of the probe inside of the drill hole when the probe is extending the entire depth of the drill hole. Using this measured length, the depth of the drill hole may be accurately determined.

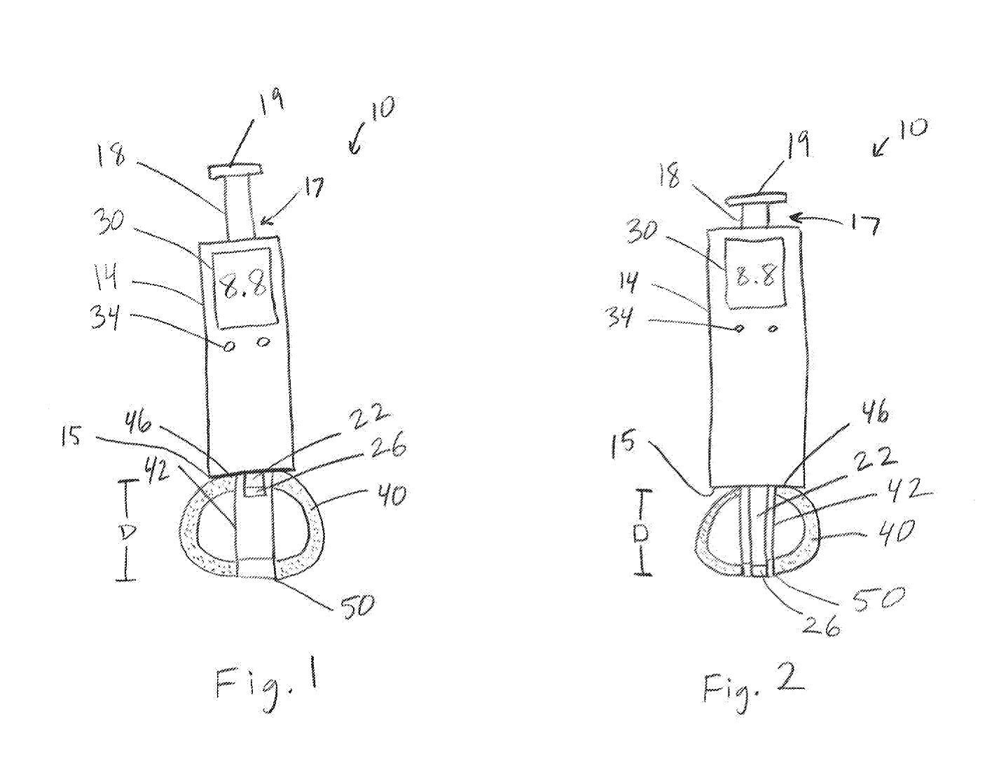

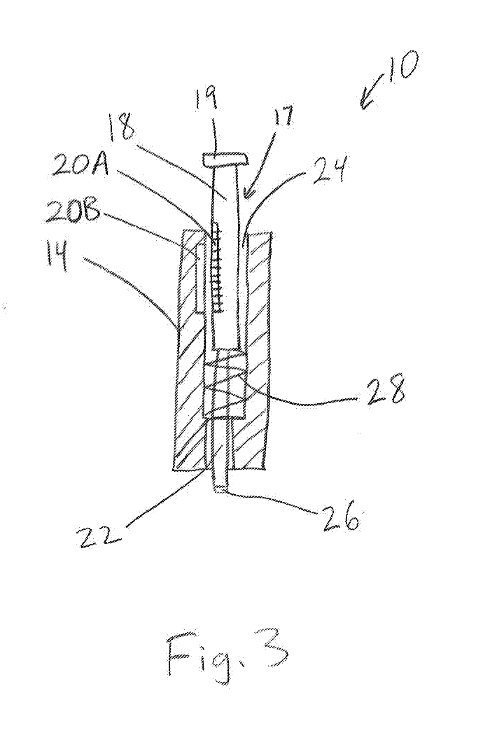

FIGS. 1-4 illustrate an embodiment of an ultrasonic depth gauge 10 in accordance with embodiments of the invention described herein. The gauge 10 includes an outer sleeve or body 14 having a rod or probe 17 including an upper portion 18 and a lower portion 22. The probe 17 is disposed within an interior cavity 24 of the outer body 14 (see FIG. 3) and is movable relative to the outer body between a retracted position shown in FIG. 1 and an extended position shown in FIG. 2. As may best be viewed in FIG. 3, a biasing member 28 (a spring in the illustrated embodiment) may be provided in the cavity 24 to bias the probe 17 in the retracted position. When a user exerts a downward force on a head portion 19 of the probe 17, the force overcomes the bias of the biasing member 28 and causes the probe to move downward (as shown in FIGS. 1-3) from the retracted position to the extended position. Although not shown, it should be appreciated that the gauge 10 may include one or more mechanisms configured to restrict the movement of the probe 17 relative to the outer body 14. For example, mechanisms or “stops” may be used to limit the distance the probe 17 may be displaced relative to the outer body 14. Further, in some embodiments, a lock may be provided to selectively fix the position of the probe 17 relative to the outer body 14. It should also be appreciated that other configurations may be provided to allow the probe 17 to be selectively displaced relative to the outer body 14 by a user, so long as the functionality described herein is achieved. Further, in some embodiments, the gauge 10 may be configured so that the rate that the probe 17 may be displaced by a user is limited, which may result in more accurate depth measurements.

The ultrasonic depth gauge 10 also includes an ultrasonic transducer 26 positioned on a bottom surface (or distal end) of the lower portion 22 of the probe 17. The ultrasonic transducer 26 is operative to emit and receive ultrasonic waves (e.g., radio or sound waves). The ultrasonic transducer 26 may comprise a single transducer or a plurality of transducers. In some embodiments, one or more transducers are used to emit ultrasonic waves and one or more different transducers are used to receive echo signals.

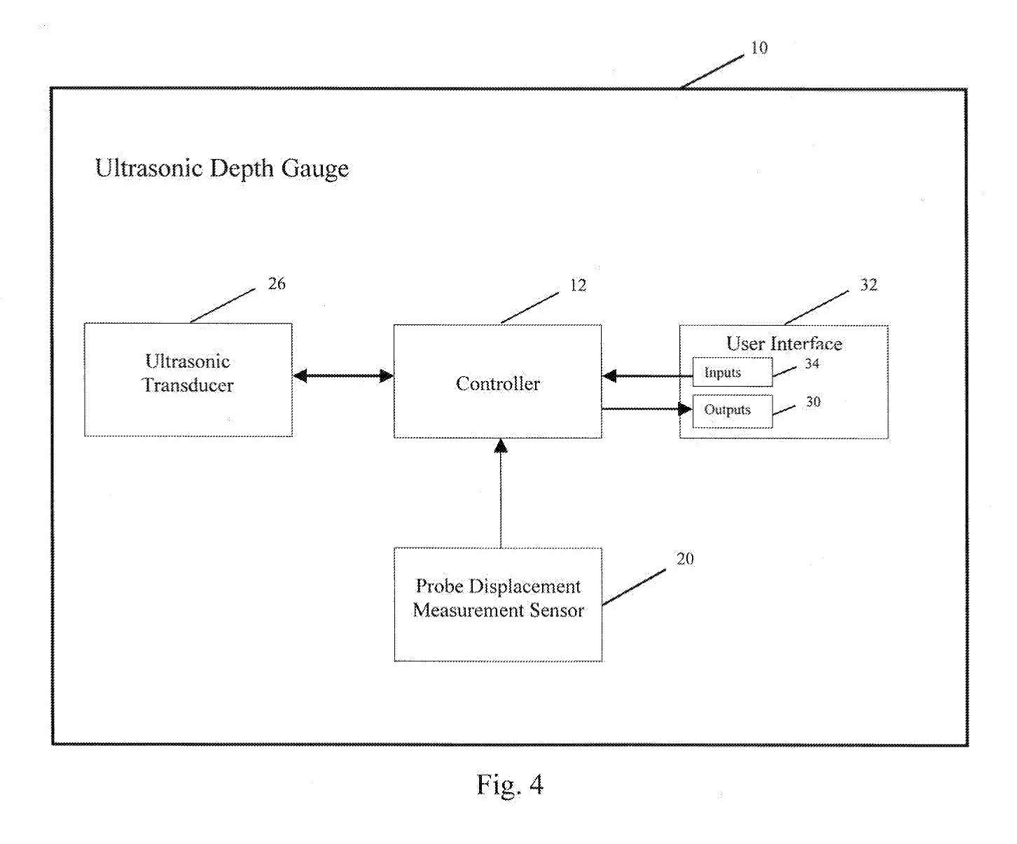

As shown in FIG. 4, the ultrasonic transducer 26 is coupled to a controller 12 operative to provide signals for the ultrasonic transducer to output and to interpret signals received from the transducer. By measuring and processing echo signals received by the ultrasonic transducer 26, the controller 12 is operative to measure one or more characteristics of the material adjacent to or surrounding the transducer 26. As an example, the controller 12 may be operative to measure the density of the material surrounding the ultrasonic transducer. At a minimum, the controller 12 is operative to detect the difference between positions wherein the ultrasonic transducer 26 is adjacent to or surrounded by a bone and positions wherein the ultrasonic transducer is adjacent to or surrounded by parts of a patient's body other than a bone (e.g., soft tissue, etc.). The controller 12 may include features of microcontrollers known in the art. For example, the controller 12 may include one or more processor cores, one or more types of memory, and input/output peripherals. The controller 12 may be application specific or a generally available controller, provided that it is capable of performing the functionality discussed herein.

The ultrasonic depth gauge 10 also includes a user interface 32 having one or more inputs (e.g., buttons or other inputs 34) and one or more outputs (e.g., a display or other outputs 30). The user interface 32 is operatively coupled to the controller 12 and allows a user to control the operation of the ultrasonic depth gauge 10. For example, such control operations may include: power on/off; reset, begin measurement, save data, change the units of measurement, etc.

As shown in FIGS. 3 and 4, the ultrasonic depth gauge 10 also includes a probe displacement measurement sensor 20 that is operatively coupled to the controller 12. In the embodiment shown in FIG. 3, the sensor 20 comprises indicia or markings 20A positioned on the upper portion 18 of the probe 17 and a scanner 20B positioned on an interior wall of the cavity 24. The scanner 20B is operative to interpret or “read” the markings 20A to determine the linear displacement of the probe 17 relative to the body 14. In some embodiments, the probe displacement measurement sensor 20 may use magnetics or optics for sensing the displacement of the probe 17. In other embodiments, the probe displacement measurement sensor 20 may utilize a mechanical system to measure displacement. It should be appreciated that any suitable technique for measuring the displacement of the probe 17 relative to the outer body 14 may be used, so long as the length of the probe extending inside a drill hole may be determined.

In operation, a user may position a bottom surface 15 of the outer body 14 against an outer surface 46 of a bone 40 such that the lower portion 22 of the probe 17 may extend into a drill hole 42 in the bone (see FIG. 1). The user may then exert a downward force on the probe 17 by pressing on the head 19 of the upper portion 18, thereby causing the probe 17 to move downward through the drill hole 42. As discussed above, other means for extending the probe 17 may be provided. As the probe 17 is moving downward, the ultrasonic transducer 26 continuously measures one or more characteristics of the material surrounding it. Since the density of the bone 40 is different from the density of the material (e.g., soft tissue) adjacent a bottom surface 50 of the drill hole 42, the ultrasonic transducer 26 is able to detect precisely the point at which it (and therefore lower portion 22 of the probe 17) reaches the bottom surface 50 of the drill hole 42.

When the controller 12 has determined that the ultrasonic transducer 26 has reached the bottom surface 50 of the drill hole 42, the controller 12 records the displacement of the probe 17 at that moment by receiving a measurement from the probe displacement measurement sensor 20. This displacement measurement corresponds to the distance between the bottom surface 15 of the outer body 14 and the bottom end of the lower portion 22 of the probe 17 (i.e., the location of the transducer 26). Since the bottom surface 15 of the outer body 14 positioned in contact with the outer surface 46 of the bone 40 at one end of the drill hole 42 and the displacement measurement is taken when the transducer 26 is at the other end of the drill hole, the displacement measurement is usable to determine the depth of the drill hole.

Once the measurement has been taken, the user may retract the probe 17 from the drill hole 42 and the controller 12 may output the depth of the drill hole 42 to the display 30 for a user to view. In some embodiments, the controller 12 may output the depth of the drill hole 42 to the display while the probe 17 is inserted, so that a user may obtain the depth reading in situ for each screw. Using this measurement, the user may then select a bone screw having an appropriate length for the drill hole 42.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the invention have been shown and described, it those skilled in the art would be aware, based upon the teachings herein, that changes and modifications may be made without departing from the embodiments described herein and their broader aspects and, therefore, the appended claims are to encompass within their scope all such changes and modifications as are within the true spirit and scope of this invention.

Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

The invention claimed is:

1. An ultrasonic depth gauge, comprising:
a probe configured to be extendable into a drill hole in a bone;
an ultrasonic transducer coupled to a distal end of the probe, the ultrasonic transducer being operative to emit ultrasonic signals and to receive echo signals;
a controller operatively coupled to the ultrasonic transducer, the controller being operative to distinguish when the ultrasonic transducer is inside a bone and when the ultrasonic transducer is not inside a bone; and
a probe displacement measurement sensor operatively coupled to the controller, the probe displacement measurement sensor being operative to measure the displacement of the probe while it is extending into the drill hole.

2. A method for measuring the depth of a drill hole in a bone, the method comprising:
inserting a probe into the drill hole, the probe including an ultrasonic transducer coupled to its distal end;
generating ultrasonic signals via the ultrasonic transducer as the probe is extended through the drill hole;
detecting echo signals via the ultrasonic transducer as the probe is extended through the drill hole;
determining when the probe has extended through the entire length of the drill hole by interpreting the echo signals; and
measuring the length of the probe that is extending through the drill hole when it is determined that the probe has extended through the entire length of the drill hole to determine the depth of the drill hole.
